(19) United States
(12) Patent Application Publication
(10) Pub. No.: US 2017/0015762 A1
(43) Pub. Date: Jan. 19, 2017

(54) CONTROLLED RADICAL POLYMERIZATION

(71) Applicant: BASF SE, Ludwigshafen (DE)

(72) Inventors: Jon Debling, Saline, MI (US); Peter Nesvadba, Marly (CH); Klaus-Dieter Hungenberg, Birkenau (DE)

(21) Appl. No.: 15/123,842

(22) PCT Filed: Mar. 4, 2015
(86) PCT No.: PCT/US2015/018710

§ 371 Date: Sep. 6, 2016

Related U.S. Application Data
(60) Provisional application No. 61/949,890, filed on Mar. 7, 2014.
(60) Provisional application No. 62/100,364, filed on Jan. 6, 2015.

(51) Int. Cl. C08F 2/38; C07D 209/46

(52) U.S. Cl. CPC C08F 2/38; C07D 209/46; C08F 2438/02

(57) ABSTRACT

A process of polymerization of a vinylic monomer uses a polymerization regulator/initiator that is a compound represented by Formula I.

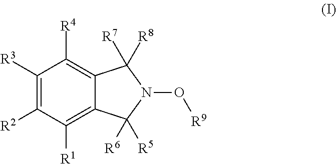

### CROSS-REFERENCE TO RELATED APPLICATIONS

This application claims priority to U.S. Provisional Application No. 62/100,364, filed Jan. 6, 2015, and U.S. Provisional Application No. 61/949,890, filed Mar. 7, 2014, the contents of which are incorporated by reference in their entirety into the present disclosure.

### FIELD

The present technology is generally related to regulating agents for controlled radical polymerization.

### BACKGROUND

Styrene/acrylic polymers are commonly made by conventional radical polymerization (RP) methods using peroxide or azo initiators. Such RP methods are conducted in a variety of process configurations over a wide range of temperatures (commonly from 50° C. to 300° C.). Using RP, the polymer chain lifetimes are very brief (fractions of seconds) and the mode of termination is uncontrolled. Uncontrolled polymerization leads to polymers with broad molecular weight distributions. Further, block copolymers are not formed when the polymerization is uncontrolled.

To address these problems, various controlled radical polymerization (CRP) techniques have been developed in the past. In typical CRP methods, a polymerization regulator is added to a composition to be polymerized, the regulator controlling the termination step and allowing the polymer chain to remain “living.” By maintaining a living character, block copolymers may be produced, and polymers with narrow molecular weight distributions may be achieved. The use of regulators such as nitroxides and regulator-initiators such as alkoxyamines for this purpose has been well established in the literature and is known as Nitroxide Mediated Polymerization (NMP). Unfortunately, existing technology for CRP uses nitroxide regulators that are limited as to the temperature range over which the process can operate, due to the thermal instability of the regulators. As a result, the current nitroxide-based CRP processes require long batch times, and have a low productivity. The art has long searched for new regulators for CRP that enable polymerization at elevated temperatures, have high productivity, which maintain the living character of the polymer, and produce polymers having narrow molecular weight distribution.

### SUMMARY

In one aspect, a process of polymerizing a vinylic monomer is provided, the process including combining a compound represented by Formula I with at least a first vinylic monomer to form a polymerization mixture; and heating the polymerization mixture to a temperature that is 130° C. or greater, and for a time sufficient to polymerize the vinylic monomer and form a first polymer; wherein Formula I is:

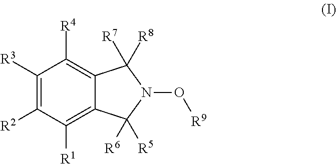

In Formula I, R¹, R², R³, and R⁴ are independently H, F, Cl, Br, I, CN, COOH, alkyl, cycloalkyl, alkoxy, alkylthio, C(O)O(alkyl), C(O)(alkyl), C(O)NH₂, C(O)NH(alkyl), C(O)N(alkyl)₂, or aryl, or R¹ and R², R² and R³, or R³ and R⁴ form together a 5- or 6-membered carbocyclic or heterocyclic ring; R⁵, R⁶, R⁷, and R⁸ are independently aryl; R⁹ is an unpaired electron, or a group which, when bearing an unpaired electron, is able to initiate radical polymerization of monomers amenable to radical polymerization. For example, R⁹ may be CR¹⁰R¹¹CN, CR¹⁰R¹¹(aryl), CR¹⁰R¹¹C(O)OH, CR¹⁰R¹¹C(O)O(alkyl), CR¹⁰R¹¹C(O)NH(alkyl), CR¹⁰R¹¹C(O)N(alkyl)₂, or CR¹⁰R¹¹C(O)(aryl) wherein each alkyl and aryl group independently at each occurrence may be substituted or unsubstituted; and R¹⁰ and R¹¹ are independently H, alkyl, or together with the carbon to which they are attached they form a 5 or 6 membered carbocyclic ring. In some embodiments, the first vinylic monomer may be a styrenic monomer, an acrylate monomer, or a methacrylate monomer. In some embodiments, where R⁹ includes aryl, aryl is phenyl or phenyl substituted with C₁-C₁₈ alkyl, O—C₁-C₁₈ alkyl, CN, —C(O)OH, —C(O)O(C₁-C₁₈ alkyl), F, Cl, Br, or I. In some embodiments, where R⁹ includes aryl, aryl is phenyl or phenyl substituted with C₁-C₁₈ alkyl, O—C₁-C₄ alkyl, CN, —C(O)OH, —C(O)O(C₁-C₄ alkyl), F, Cl, Br, or I.

The first polymer may be a first living polymer. Accordingly, adding at least a second vinylic monomer, either together with the first, or sequentially to the first, will result in a copolymer or block copolymer, respectively. Similarly, adding at least a third vinylic monomer, either together with the first and second, or sequentially to the first and second, will result in a terpolymer that is a copolymer, or block copolymer, respectively.

In another aspect, the polymers formed by any of the above process are provided.

In another aspect, a composition including any of the above polymers is provided. The compositions may include any one or more of the following: an adhesive, coating, plasticizer, pigment dispersant, compatibilizer, tackifier, surface primer, binder, or chain extender.

In another aspect, a compound is provided represented by Formula I:

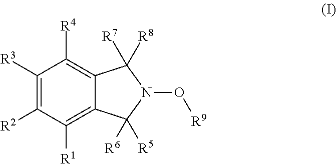

With regard to the compound as represented by Formula I, R¹, R², R³, and R⁴ are independently H, F, Cl, Br, I, CN, C(O)OH, alkyl, cycloalkyl, alkoxy, alkylthio, C(O)O(alkyl), C(O)(alkyl), C(O)NH₂, C(O)NH(alkyl), C(O)N(alkyl)₂, or aryl, or R¹ and R², R² and R³, or R³ and R⁴ form together a 5- or 6-membered carbocyclic or heterocyclic ring; R⁵, R⁶, R⁷, and R⁸ are independently aryl; R⁹ is an unpaired electron, or a group which, when bearing an unpaired electron, is able to initiate radical polymerization of monomers amenable to radical polymerization. For example, R⁹ may be CR¹⁰R¹¹CN, CR¹⁰R¹¹(aryl), CR¹⁰R¹¹C(O)OH, CR¹⁰R¹¹C(O)O(alkyl), CR¹⁰R¹¹C(O)NH(alkyl), CR¹⁰R¹¹C(O)N(alkyl)₂, or CR¹⁰R¹¹C(O)(aryl); and R¹⁰ and R¹¹ are independently H, alkyl, or together with the carbon to which they are attached they form a 5 or 6 membered carbocyclic ring. In some embodiments, where R⁹ includes aryl, aryl is phenyl or phenyl substituted with C₁-C₁₈ alkyl, O—C₁-C₁₈ alkyl, CN, —C(O)OH, —C(O)O(C₁-C₁₈ alkyl), F, Cl, Br, or I. In some embodiments, where R⁹ includes aryl, aryl is phenyl or phenyl substituted with C₁-C₁₈ alkyl, O—C₁-C₄ alkyl, CN, —C(O)OH, —C(O)O(C₁-C₄ alkyl), F, Cl, Br, or I. However, with respect to the compound itself it is subject to the proviso that where R¹, R², R³, and R⁴ are H and R⁹ is an unpaired electron or CHCH₃Ph, at least one of R⁵, R⁶, R⁷, and R⁸ is other than unsubstituted phenyl, or where R⁹ is an unpaired electron or CHCH₃Ph, and R⁵, R⁶, R⁷, and R⁸ are unsubstituted phenyl, at least one of R¹, R², R³, and R⁴ is other than H, and where R⁹ is an unpaired electron or CHCH₃Ph, and three of R⁵, R⁶, R⁷, and R⁸ are unsubstituted phenyl and one of R⁵, R⁶, R⁷, and R⁸ is methyl, at least one of R¹, R², R³, and R⁴ is other than H.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D are graphs presenting data from testing of a batch NMP of styrene at 160° C. using an alkoxyamine, according to Example 4. FIG. 1 A is a graph of the conversion of the styrene to a polymer versus time. FIG. 1B is a graph of the normalized conversion versus time. FIG. 1C is a graph of the number average molecular weight verses conversion. FIG. 1D is a graph of the polydispersity index (PDI) versus conversion. Molar ratios of the [Alk]:[Sty] of 1:25; 1:50; 1:100; and 1:300 were used in the graph. The thermal polymerization profile is included for comparison.

FIGS. 2A-2D are graphs presenting data from testing of a batch NMP of styrene at various reaction temperatures using an alkoxyamines, according to Example 4. FIG. 2A is a graph of the conversion of the styrene to a polymer versus time. FIG. 2B is a graph of the normalized conversion versus time. FIG. 2C is a graph of the number average molecular weight verses conversion. FIG. 2D is a graph of the polydispersity index (PDI) versus conversion. The experiments were conducted at 140° C.; 160° C.; 180° C.; and 200° C. with a molar ratio of the [Alk]:[Sty] for all examples in FIGS. 2A-2D of 1:50.

FIG. 3 is a graph of chain extension of polystyrene (pSTY) at 180° C., according to Example 4. The number average molecular weight evolved from 3160 g/mol to 23,060 g/mol after 20 minutes of reaction.

FIG. 4 is a graph of the molar mass distributions measured for the alkoxyamine-mediated batch polymerization of bulk styrene at 200° C.; the initial molar ratio of alkoxyamine to styrene is 1:50 (DPn=50). The reaction time, conversion and polydispersity index (PDI) are presented in the legend.

FIG. 5 is a graph of the molar mass distribution resulting from chain extension of polystyrene by bulk NMP at 160° C. The reaction times and conversions are presented in the legend.

FIGS. 6A-B are graphs of the batch NMP of butyl acrylate in 50% v/v dimethylformaide (DMF) with an alkoxyamine at various reaction temperatures (see legend) under nitrogen (<1 atm). FIG. 6A provides the conversion versus time. FIG. 6B provides the number-average molar mass (Mₙ; closed symbols) on the left-hand y-axis and the polydispersity index ( [symbol] ; open symbols) on the right-hand y-axis, both with respect to conversion (x-axis). The initial molar ratio of alkoxyamine:butyl acrylate is 1:50 for all examples in FIGS. 6A-B.

FIGS. 7A-B are graphs of the batch NMP of styrene at 160° C., with initial alkoxyamine:styrene molar ratios presented in the legend. FIG. 7A provides the conversion versus time. FIG. 7B provides the number-average molar mass (Mₙ; closed symbols) on the left-hand y-axis and the polydispersity index ( [symbol] ; open symbols) on the right-hand y-axis, both with respect to conversion (x-axis). The thermal polymerization profile at 160° C. (“Target” line) is included for comparison.

FIGS. 8A-B are graphs of batch NMP of bulk styrene by an alkoxyamine of the present technology at various reaction temperatures, with an initial alkoxyamine:styrene molar ratio of 1:50. FIG. 8A provides the conversion versus time. FIG. 8B provides the number-average molar mass (Mₙ; closed symbols) and dispersity ( [symbol] ; open symbols) versus conversion. The thermal polymerization profile at 200° C. (“Target” line) is included for comparison.

FIGS. 9A-B are graphs of the batch NMP of butyl acrylate at various reaction temperatures, with an initial alkoxyamine:butyl acrylate molar ratio of 1:55. FIG. 9A provides the conversion versus time. FIG. 9B provides number-average molar mass (Mₙ; closed symbols) and dispersity ( [symbol] ; open symbols) versus conversion.

FIGS. 10A-B are graphs of the molar mass distribution resulting from bulk NMP at 160° C. of styrene (FIG. 10A) and butyl acrylate (FIG. 10B) using an alkoxyamine of the present technology with initial alkoxyamine:monomer molar ratios of 1:50 (styrene) and 1:55 (butyl acrylate). Polymerization time and conversion presented in the legend and discussed in the Examples.

FIGS. 11A-B are graphs of the batch NMP of butyl acrylate (BA) at 160° C., with initial alkoxyamine:butyl acetate molar ratios presented in the legend. FIG. 11A provides the conversion versus time. FIG. 11B provides the number-average molar mass (Mₙ; closed symbols) and dispersity ( [symbol] ; open symbols) versus conversion.

FIGS. 12A-B are graphs of batch NMP of styrene (STY), a 50:50 molar ratio of acrylic acid:styrene (AA:STY), and a 90:10 molar ratio of butyl methacrylate:styrene (0.9BMA:0.1STY) at 160° C. utilizing an alkoxyamine of the present technology, where the initial alkoxyamine:monomer molar ratio was 1:50. FIG. 12A provides the conversion versus time for each of these systems. FIG. 12B provides the number-average molar mass (Mₙ; closed symbols) and dispersity ( [symbol] ; open symbols) versus conversion for each system. FIG. 12B also provides the thermal polymerization profile at 160° C. (“Target” lines) for each system for comparison.

FIGS. 13A, 13B, and 13C show the rate of reaction at 160° C. (13A) and 180° C. (13B), and the molecular weight distribution (13C) for a comparative example using TEMPO as a catalyst, according to the comparative example.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

In general, “substituted” refers to an alkyl, alkenyl, alkynyl, aryl, or ether group, as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

As used herein, “alkyl” groups include straight chain and branched alkyl groups having from 1 to about 20 carbon atoms, and typically from 1 to 12 carbons or, in some embodiments, from 1 to 8 carbon atoms. As employed herein, “alkyl groups” include cycloalkyl groups as defined below. Alkyl groups may be substituted or unsubstituted. Examples of straight chain alkyl groups include methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, neopentyl, and isopentyl groups. Representative substituted alkyl groups may be substituted one or more times with, for example, amino, thio, hydroxy, cyano, alkoxy, and/or halo groups such as F, Cl, Br, and I groups. As used herein the term haloalkyl is an alkyl group having one or more halo groups. In some embodiments, haloalkyl refers to a per-haloalkyl group.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups may be substituted or unsubstituted. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, but not limited to: 2,2-; 2,3-; 2,4-; 2,5-; or 2,6-disubstituted cyclohexyl groups or mono-, di-, or tri-substituted norbornyl or cycloheptyl groups, which may be substituted with, for example, alkyl, alkoxy, amino, thio, hydroxy, cyano, and/or halo groups.

Alkenyl groups are straight chain, branched or cyclic alkyl groups having 2 to about 20 carbon atoms, and further including at least one double bond. In some embodiments alkenyl groups have from 1 to 12 carbons, or, typically, from 1 to 8 carbon atoms. Alkenyl groups may be substituted or unsubstituted. Alkenyl groups include, for instance, vinyl, propenyl, 2-butenyl, 3-butenyl, isobutenyl, cyclohexenyl, cyclopentenyl, cyclohexadienyl, butadienyl, pentadienyl, and hexadienyl groups among others. Alkenyl groups may be substituted similarly to alkyl groups. Divalent alkenyl groups, i.e., alkenyl groups with two points of attachment, include, but are not limited to, CH—CH═CH₂, C═CH₂, or C═CHCH₃.

As used herein, “aryl”, or “aromatic,” groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. The phrase “aryl groups” includes groups containing fused rings, such as fused aromatic-aliphatic ring systems (e.g., indanyl, tetrahydronaphthyl, and the like). Aryl groups may be substituted or unsubstituted. As used herein, the terms alkylphenyl and alkylnaphthyl refer to phenyl and naphthyl groups that have one or more alkyl groups on the ring.

It has now been found that certain nitroxides may be used as regulators and the related alkoxyamines as initiators-regulators for elevated temperature controlled radical polymerizations (ETCRP). The nitroxides are stable up to high temperatures and provide for controlled polymerization. As used herein the term “regulator” refers to the ability of the material, in this case the nitroxide, to control the termination step of the polymerization and allow the forming polymer to remain “living.” That is, it allows the forming polymer to accept additional monomer or monomers, until the polymerization is intentionally terminated. In some embodiments, the nitroxide regulators are stable up to temperatures of 200° C., or greater.

The alkoxyamines may be generally represented by Formula I:

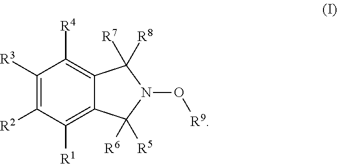

In Formula I, R¹, R², R³, and R⁴ are each independently H, F, Cl, Br, I, CN, COOH, alkyl, cycloalkyl, alkoxy, alkylthio, C(O)O(alkyl), C(O)(alkyl), C(O)NH₂, C(O)NH(alkyl), C(O)N(alkyl)₂, or aryl, or R¹ and R², R² and R³, or R³ and R⁴ form together a 5- or 6-membered carbocyclic or heterocyclic ring; R⁵, R⁶, R⁷, and R⁸ are independently aryl; R⁹ is an unpaired electron, CR¹⁰R¹¹CN, CR¹⁰R¹¹(aryl), CR¹⁰R¹¹C(O)OH, CR¹⁰R¹¹C(O)O(alkyl), CR¹⁰R¹¹C(O)NH(alkyl), CR¹⁰R¹¹C(O)N(alkyl)₂, or CR¹⁰R¹¹C(O)(aryl) wherein each alkyl and aryl group independently at each occurrence may be substituted or unsubstituted; and R¹⁰ and R¹¹ are independently H, alkyl, or together with the carbon to which they are attached they form a 5 or 6 membered carbocyclic ring. In any of the above embodiments, it may be that the aryl group of R¹, R², R³, R⁴, R⁵, R⁶, R⁷, or R⁸ is independently phenyl or naphthyl. Any of the described aryl or alkyl groups of R⁹ may be optionally substituted with one or more C₁-C₁₈ alkyl, O(C₁-C₁₈ alkyl), OH, CN, C(O)OH, C(O)O(C₁-C₁₈ alkyl), F, Cl, Br, or I. For example, any of the described aryl or alkyl groups of R⁹ may be optionally substituted with one or more C₁-C₁₈ alkyl, O(C₁-C₄ alkyl), OH, CN, C(O)OH, C(O)O(C₁-C₄ alkyl), F, Cl, Br, or I. In any of the above embodiments, it may be that where R⁹ includes aryl, aryl is phenyl (Ph) or phenyl substituted with C₁-C₁₈ alkyl, O—C₁-C₁₈ alkyl, CN, —C(O)OH, —C(O)O(C₁-C₁₈ alkyl), F, Cl, Br, or I. In any of the above embodiments, it may be that the one or more of the phenyl or alkyl groups of R⁹ are independently substituted with one C₁-C₁₈ alkyl, O(C₁-C₄ alkyl), OH, CN, C(O)OH, C(O)O(C₁-C₄ alkyl), F, Cl, Br, or I. The stable nitroxide compounds may be generally represented by Formula I where R⁹ is an unpaired electron.

In some embodiments of the compound of Formula I, R¹, R², R³, and R⁴ are independently H, F, Cl, Br, I, CN, COOH, C₁-C₆ alkyl, C₅-C₆ cycloalkyl, C₁-C₆ alkoxy, C₁-C₆ alkylthio, C(O)O(C₁-C₆ alkyl), C(O)(C₁-C₆ alkyl), C(O)NH₂, C(O)NH(C₁-C₆ alkyl), C(O)N(C₁-C₆ alkyl)₂, or phenyl, or R¹ and R², R² and R³, or R³ and R⁴ form together a 5- or 6-membered carbocyclic or heterocyclic ring. In some embodiments of the compound of Formula I, R⁵, R⁶, R⁷, and R⁸ are independently phenyl, naphthyl, alkylphenyl, or alkylnaphthyl. In some embodiments of the compound of Formula I, R⁹ is an unpaired electron (i.e. a stable nitroxide), CH₂Ph, C(CH₃)₂CN, CH(CH₃)Ph, C(CH₃)₂Ph, CR¹⁰R¹¹C(O)OH, CR¹⁰R¹¹C(O)O(C₁-C₆ alkyl), CR¹⁰R¹¹C(O)(C₁-C₆ alkyl), CR¹⁰R¹¹C(O)NH(C₁-C₆ alkyl), or CR¹⁰R¹¹C(O)N(C₁-C₆ alkyl)₂ wherein each alkyl and Ph group independently at each occurrence may be substituted or unsubstituted; and R¹⁰ and R¹¹ are independently H, C₁-C₄ alkyl, or form together with the carbon to which they are attached a 5- or 6-membered carbocyclic ring. In some embodiments of the compound of Formula I, R¹, R², R³, and R⁴ are hydrogen; R⁵, R⁶, R⁷, and R⁸ are phenyl, naphthyl, alkylphenyl, or alkylnaphthyl; CH₂Ph, C(CH₃)₂CN, CH(CH₃)Ph, C(CH₃)₂Ph, CR¹⁰R¹¹C(O)OH, CR¹⁰R¹¹C(O)O(C₁-C₆ alkyl), CR¹⁰R¹¹C(O)(C₁-C₆ alkyl), CR¹⁰R¹¹C(O)NH(C₁-C₆ alkyl), or CR¹⁰R¹¹C(O)N(C₁-C₆ alkyl)₂ wherein each alkyl and Ph group independently at each occurrence may be substituted or unsubstituted; and R¹⁰ and R¹¹ are independently H, or CH₃. In some embodiments of the compound of Formula I, R¹, R², R³, and R⁴ are hydrogen; R⁵, R⁶, R⁷, and R⁸ are phenyl; R⁹ is an unpaired electron, CH(CH₃)Ph, CR¹⁰R¹¹C(O)OH, CR¹⁰R¹¹C(O)O(C₁-C₆ alkyl), CR¹⁰R¹¹C(O)NH(C₁-C₆ alkyl), or CR¹⁰R¹¹C(O)N(C₁-C₆ alkyl)₂ wherein each alkyl group independently at each occurrence may be substituted or unsubstituted and wherein each Ph group may independently at each occurrence be unsubstituted or substituted with one or more C₁-C₁₈ alkyl, O(C₁-C₄ alkyl), CN, C(O)OH, C(O)O(C₁-C₄ alkyl), or halogen groups; and R¹⁰ and R¹¹ are independently H, or CH₃. In a preferred embodiment of the compound of Formula I, R¹, R², R³, and R⁴ are hydrogen; R⁵, R⁶, R⁷, and R⁸ are phenyl; R⁹ is an unpaired electron, CH(CH₃)Ph, CR¹⁰R¹¹C(O)OH, CR¹⁰R¹¹C(O)O(C₁-C₆ alkyl), CR¹⁰R¹¹C(O)NH(C₁-C₆ alkyl), or CR¹⁰R¹¹C(O)N(C₁-C₆ alkyl)₂ wherein each alkyl group independently at each occurrence may be substituted or unsubstituted and wherein each Ph group may independently at each occurrence be unsubstituted or substituted with one or more C₁-C₁₈ alkyl, O(C₁-C₄ alkyl), CN, C(O)OH, C(O)O(C₁-C₄ alkyl), or halogen groups; and R¹⁰ and R¹¹ are independently H, or CH₃. In any of the above embodiments, it may be that R⁹ is

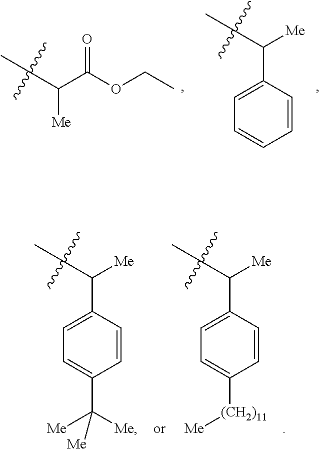

Provided herein are alkoxyamines and nitroxides, and processes of using such compounds as polymerization initiators-regulators (alkoxyamines) and/or regulators (nitroxides). Any of the above compounds of Formula I may be used in such processes. Where just the isolated compounds are described (i.e. not with regard to the methods necessarily), the compound of Formula I may be subject to the proviso that where R¹, R², R³, and R⁴ are H and R⁹ is an unpaired electron or C(H)(CH₃)Ph, at least one of R⁵, R⁶, R⁷, and R⁸ is other than unsubstituted phenyl, or where R⁹ is an unpaired electron or C(H)(CH₃)Ph, and R⁵, R⁶, R⁷, and R⁸ are unsubstituted phenyl, at least one of R¹, R², R³, and R⁴ are other than H, and where R⁹ is an unpaired electron or C(H)(CH₃)Ph, and three of R⁵, R⁶, R⁷, and R⁸ are unsubstituted phenyl and one of R⁵, R⁶, R⁷, and R⁸ is methyl, at least one of R¹, R², R³, and R⁴ is other than H.

As will be appreciated, the compound of Formula I describes a unimolecular alkoxyamine initiator-regulator if R⁹ is a group which, when bearing an unpaired electron, is able to initiate radical polymerization of monomers amenable to radical polymerization. The formation of the regulating nitroxide radical and the initiating radical R⁹ from the unimolecular alkoxyamine initiator-regulator of Formula I occurs according to the following scheme

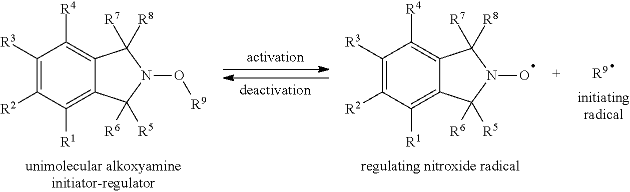

The activation leading to the above homolytic cleavage can occur thermally or photochemically.

As noted, the compounds of Formula I may be used in a polymerization process. The process includes preparation of homopolymers, co-polymers, and block polymers. The monomers used in the polymerization process are typically vinylic monomers that are amenable to radical polymerizations. The process includes combining any one or more of the compounds represented by Formula I, above, with a first vinylic monomer to form a polymerization mixture, and heating the polymerization mixture a temperature, and for a time, sufficient to polymerize the vinylic monomer and form a first polymer. The first polymer may be the desired polymer, in which case, termination of the polymer may be effected, and the first polymer obtained. The first vinyl monomer may be a single type of monomer, such that the first polymer formed is a homopolymer. Alternatively, the first vinylic monomer may be a mixture of monomers, in which case the first polymer formed is a random, gradient or alternating co-polymer. The polymerization may be terminated simply by cooling the polymerization mixture.

However, further, sequential polymerization may be conducted to form polymers with other properties. As noted above, the regulators described above provide for living polymerizations at the temperatures used. That is, a second monomer (or mixture of monomers) may be added to the first polymer to form block co-polymers. Alternating addition of the first monomer(s) and second monomer(s) results in the formation of blocks of the first and second monomers, or additional monomer blocks (third, fourth, fifth . . . ) as the case may be. The most recently added monomers build upon the polymers formed in the previous step.

Vinylic monomers for use in the process of forming polymers include, but are not limited to, styrenic monomers, acrylate monomers, and methacrylate monomers. Illustrative vinylic monomers include, but are not limited to, styrene, α-methylstyrene, N-vinylpyrrolidone, 4-vinylpyridine, vinyl imidazole, butyl acrylate, butyl methacrylate, ethyl acrylate, ethyl methacrylate, 2-ethyl hexyl acrylate, 2-ethyl hexyl methacrylate, methyl methacrylate, vinyl acetate, methyl acrylate, 2-hydroxyethyl methacrylate, 2-hydroxyethyl acrylate, glycidyl acrylate, glycidyl methacrylate, propyl acrylate, propyl methacrylate, (polyethylene glycol) methyl ether acrylate, (polyethylene glycol) methyl ether methacrylate, acrylic acid, methacrylic acid, itaconic acid, maleic acid, fumaric acid, crotonic acid, acrylonitrile, acryl amide, N-isopropylacrylamide, methacrylamide, vinyl acetate, or vinyl chloride. Homopolymers are formed where only a single type of vinylic monomer is used, and co-polymers may be formed where more than one type of vinylic monomer is used. Block co-polymers may also be formed using two or more vinylic monomers, as further described below.

In the process, the temperature and time are sufficient to effect polymerization of the vinylic monomer(s). The processes are particularly amenable to regulating and controlling polymerizations at elevated temperatures. For example, the temperature may be 130° C. or greater. This includes, in some embodiments, the temperature being from about 130° C. to about 240° C., inclusive. In other embodiments, the temperature is about 150° C. to about 160° C. In further embodiments, the temperature is about 160° C. to about 200° C. With regard to the time of the polymerization, it may be from about 5 minutes to about 240 minutes. In some embodiments, this includes from about 5 minutes to about 60 minutes. In some embodiments, this includes from about 15 minutes to about 30 minutes.

In the process, the amount of the alkoxyamine, or regulator/initiator, may be varied. This amount may be expressed as a ratio of the compound of Formula I to the vinylic monomer. The ratio may be from about 1:10 to about 1:500 on a mol basis. The ratio may be from about 1:25 to about 1:300 on a mol basis. In some embodiments, the ratio is from about 1:50 to about 1:200 on a mol basis.

In the process, where monomers are used that undergo thermal auto-initiation, the nitroxide regulator (i.e. a compound of Formula I where R⁹ is an unpaired electron) may be used in conjunction with the auto-initiating monomer, without the presence of the alkoxyamine initiator-regulator (i.e. a compound of Formula I, where R⁹ is other than the unpaired electron). Illustrative auto-initiating monomers include, but are not limited to, styrenic monomers such as styrene and α-methylstyrene.

The process, may also include adding a radical initiator in addition to a compound of Formula I. Illustrative radical initiators that may also be used include, but are not limited to, peroxides or azo-initiators. For example, the radical initiator may be 2,2′-azodi-(2,4-dimethylvaleronitrile), 2,2′-azobisisobutyronitrile (AIBN), 2,2′-azobis-(2-methylbutyronitrile), 1,1′-azobis (cyclohexane-1-carbonitrile), tert-butylperbenzoate, tert-amyl peroxy-2-ethylhexyl carbonate, 1,1-bis(tert-amylperoxy)cyclohexane, tert-amylperoxy-2-ethylhexanoate, tert-amylperoxyacetate, tert-butylperoxyacetate, tert-butylperoxybenzoate (TBPB), 2,5-di-(tert-butylperoxy)-2,5-dimethylhexane, di-tert-amyl peroxide (DTAP), di-tert-butylperoxide (DTBP), lauryl peroxide, dilauryl peroxide (DLP), succinic acid peroxide; or benzoyl peroxide.

In some embodiments, rate accelerating additives may be added to accelerate the polymerization. Illustrative examples include, but are not limited to, benzoic acid, p-toluenesulfonic acid, acetic anhydride, trifluoroacetic acid anhydride, malononitrile, acetylacetone, acetoacetic esters, or diethyl malonate.

In some embodiments, a mixture of the alkoxyamine initiator-regulator with the nitroxide regulator may be used. In such embodiments, the ratio of alkoxyamine:nitroxide may be from about 200:1 to about 100:10.

The process may be conducted using a wide variety of reactor types and may be set up in a continuous, batch, or semi-batch configuration. Such reactors include, but are not limited to, continuous stirred tank reactors (“CSTRs”), batch reactors, semi-batch reactors, tube reactors, loop reactors, or in a reactor system that is a combination of any two or more such reactors. For example, in one embodiment, the process is conducted in a batch reactor, a continuous stirred tank reactor, a series of two or more continuous stirred tank reactors, a loop reactor, a series of two or more loop reactors, a semi-batch reactor, or a combination of any two or more such reactors. In another embodiment, the process is conducted in a continuous stirred tank reactor, or series of two or more continuous stirred tank reactors.

Where 2 or more reactors are combined in series, pre-polymerization may be conducted of a monomer in a first reactor to form a living polymer. The living polymer may then be fed to a second reactor where the living polymer is further polymerized either with the same monomer or a different monomer. Where different monomers are used, a block co-polymer may be formed. Further blocks may be added with additional monomers in subsequent reactors.

In another aspect, the polymers are provided that are formed by any of the above processes using any of the above compounds of Formula I. For example, the first polymer may be provided with is a homopolymer or a random co-polymer, or the block co-polymers of two or more vinylic monomers may be provided.

Depending upon the monomers used, the temperatures used, and the duration of the polymerization, the formed polymers may have a wide range of molecular weights. For example, the polymers may have a number average molecular weight of from about 500 Daltons to about 100,000 Daltons. In some embodiments, the number average molecular weight is from about 500 Daltons to about 25,000 Daltons. In some embodiments, the number average molecular weight is from about 500 Daltons to about 2,500 Daltons. The polymers produced may also exhibit a glass transition from about −70° C. to about 140° C. In some embodiments, the glass transition temperature is from about 0° C. to about 100° C.

The regulators control the polymerization process and allow for the production of polymers having a consistent polydispersity index (PDI; [symbol] ). That is, a relatively consistent molecular weight distribution is achieved through radical polymerizations employing the compounds of Formula I. For example, the polymers formed by the process may exhibit a PDI from about 1.1 to about 1.8. In some embodiments, the polymers formed by the process may exhibit a PDI from about 1.1 to about 1.7. In some embodiments, the polymers formed by the process may exhibit a PDI from about 1.1 to about 1.6. In some embodiments, the polymers formed by the process may exhibit a PDI from about 1.1 to about 1.5. In some embodiments, the polymers formed by the process may exhibit a PDI from about 1.1 to about 1.4. In some embodiments, the polymers formed by the process may exhibit a PDI from about 1.2 to about 1.4.

In another aspect, compositions that include the polymers are also provided. For example, such compositions may include the polymer with any one or more of crosslinking agents, solvents, pigments, curing agents, dispersion agents, surfactants, leveling agents, drying agents, and/or other additives. Such compositions may be useful as an adhesive, coating, plasticizer, pigment dispersant, compatibilizer, tackifier, surface primer, binder, or chain extender.

The compounds of Formula I, the processes of polymerization employing the compounds of Formula I and the polymers prepared therefrom provide some distinct advantages over non-regulated radical polymerizations. For example, the stable regulators permits controlled radical polymerizations at elevated temperatures. The polymers formed have relatively narrow molecular weight distributions, and block structures can be produced much more efficiently and with lower cost than conventional controlled polymerization processes. Finally, such composition of the polymers provide for new coatings, adhesives, plasticizers, pigment dispersants, compatibilizers, tackifiers, surface primers, binders, and chain extenders.

The present invention, thus generally described, will be understood more readily by reference to the following examples, which are provided by way of illustration and are not intended to be limiting of the present invention.

### Examples

In the following examples, monomer conversions were determined by ¹H NMR analysis using a Bruker Avance-400 (400 MHz) instrument after adding deuterated chloroform (Aldrich).

Where applicable, size exclusion chromatography (SEC) was performed using a Waters 2960 GPC separation module with Styragel packed columns HR 0.5, HR 1, HR 3, HR4, and HR 5E (Waters Division Millipore). Using distilled tetrahydrofuran (THF) as eluent at 0.3 mL/minute, the detection was provided by a Waters 410 Differential Refractometer, and Wyatt Instruments Dawn EOS 690 nm laser photometer multiangle light scattering (LS) unit. The detector was calibrated with eight narrow polystyrene standards, ranging from 374 to 355,000 g/mol. The molecular weights of poly(BA), poly(BMA), and poly(AA) samples were obtained by universal calibration using known Mark-Houwink parameters for polystyrene (K=11.4×10⁻⁵ dL/g, a=0.716), poly(BMA) (K=14.8×10⁻⁵ dL/g, a=0.664), poly(BA) (K=7.4×10⁻⁵ dL/g, a=0.750), and poly(MA) (K=9.5×10⁻⁵ dL/g, a=0.719).

Poly(acrylic acid) (“poly(AA)”) samples were methylated before SEC analysis to ensure solubility in THF. Poly(AA) was first solubilized in a mixture of methanol and THF at room temperature. The methylating agent trimethylsilyldiazomethane was added dropwise to the polymer solution until no bubbling is witnessed and the solution remains yellow in color, indicating full conversion to the methyl ester with excess methylating agent.

General.

1,3-Dihydro-1,1,3,3-tetraphenyl-2-(1-phenylethoxy)-1H-isoindol was prepared as described in WO 2001/092228. The structure of the compound is:

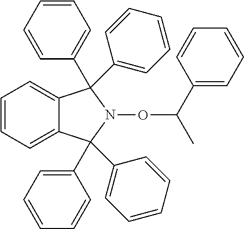 [SMILES: CC(ON1C(c2ccccc2)(c2ccccc2)c2ccccc2C1(c1ccccc1)c1ccccc1)c1ccccc1]

### Example 1

### Preparation of ethyl 2-((1,1,3,3-tetraphenylisoindolin-2-yl)oxy)propanoate

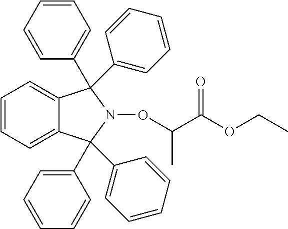 [SMILES: CCOC(=O)C(C)ON1C(c2ccccc2)(c2ccccc2)c2ccccc2C1(c1ccccc1)c1ccccc1]

A 50 ml flask was filled with argon and charged with dichloromethane (15 ml), 1,1,3,3-tetraphenylisoindoline-N-oxyl (2.19 g, 5 mmol, prepared as described in WO 2001/092228), ethyl-2-bromopropionate (1.36 g, 7.5 mmol) and copper(I) bromide (2.15 g, 15 mmol). Within 15 minutes, and at room temperature, a solution of N,N,N′,N′,N″-pentamethyldiethylene-triamine (2.60 g, 15 mmol) in absolute ethanol (6 ml) is added. The resulting green suspension is stirred for 26 hours under argon. Water (50 ml) is then added and the mixture is extracted with dichloromethane (3×30 ml). The combined extracts were washed with water (20 ml), 1M-HCl (2×20 ml), 1M-NH₃ (20 ml) and water (20 ml) and dried over MgSO4. The solid residue (2.9 g) was crystallized from dichloromethane-heptane to afford 2.21 g of the title compound as a white crystals, mp. 207-211° C. ¹H-NMR (400 MHz, CDCl₃, δ ppm): 7.6-6.7 (m, 24 ArH), 4.49-4.44 (q, J=5.4 Hz, —OCH(CH₃)), 3.73-3.65 (m, 1H, O—CHₐH_{b}CH₃), 3.51-3.43 (m, 1H, O—CHₐH_{b}CH₃), 1.07-0.99 (m, 2×CH₃).

### Example 2

### Preparation of 2-[1-(4-dodecylphenyl)ethoxy]-1,1,3,3-tetraphenyl-isoindoline

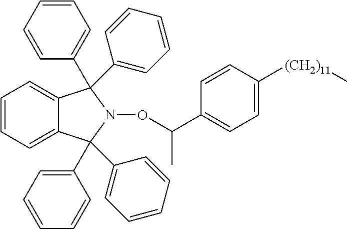

A) Synthesis of the intermediate 1-(1-Bromoethyl)-4-dodecylbenzene. To a solution of 1-(1-hydroxyethyl)-4-dodecylbenzene (9.3 g, 32 mmol, prepared as described by Y. Yang et al., Journal of the American Chemical Society, 134(36), 14714-14717; 2012) in 100 ml of dichloromethane is, at room temperature, added phosphorus tribromide (10.08 g, 37 mmol). After 4 hours acetyl bromide (6.44 g, 52 mmol) was also added. The slightly yellow solution was stirred for 120 hours at room temperature, then washed with cold water (3×50 ml), 1M-NaHCO₃ (3×50 ml), dried over MgSO₄, and evaporated to afford 10.4 g of 1-(1-bromoethyl)-4-dodecylbenzene as a slightly yellow oil. ¹H-NMR (400 MHz, CDCl₃, δ ppm): 7.39-7.37 (d, 2 ArH), 7.19-7.17 (d, 2 ArH), 5.29-5.23 (q, CHCH₃), 2.64-2.60 (t, CH₂), 2.09-2.07 (d, CHCH₃), 1.65-1.60 (m, CH₂), 1.34-1.30 (m, 9×CH₂), 0.93-0.91 (t, CH₃).

B) 2-[1-(4-Dodecylphenyl)ethoxy]-1,1,3,3-tetraphenyl-isoindoline. A 100 ml flask was filled with argon and charged with dichloromethane (15 ml), 1,1,3,3-tetraphenylisoindoline-N-oxyl (2.19 g, 5 mmol, prepared as described in WO 2001/092228), 1-(1-bromoethyl)-4-dodecylbenzene (2.65 g, 7 mmol) and copper(I) bromide (2.15 g, 15 mmol). To the stirred suspension was added a solution of N,N,N′,N′,N″-pentamethyldiethylene-triamine (2.60 g, 15 mmol) in absolute ethanol (6 ml). The resulting green suspension was stirred for 18 hours under argon. Water (50 ml) was then added and the mixture extracted with dichloromethane (3×30 ml). The combined extracts were washed with water (20 ml), 1M-HCl (2×20 ml), 1M-NH₃ (20 ml), and water (20 ml), and dried over MgSO₄. The residue (4.4 g) was chromatographed on silica gel with heptane-ethyl acetate (50:1) and the pure fractions were crystallized from dichloromethane-acetonitrile to afford 2.25 g of the title compound as a white crystals, mp. 42-47° C. ¹H-NMR (400 MHz, CDCl₃, δ ppm): 7.6-6.7 (m, 28 ArH), 4.75-4.70 (q, J=4.8 Hz, —CHCH₃), 2.56-2.52 (t, CH₂), 1.70-0.90 (m, —CHCH₃+(CH₂)₁₀CH₃)

### Example 3

### Preparation of 2-[1-(4-tert-butylphenyl)ethoxy]-1,1,3,3-tetraphenyl-isoindoline

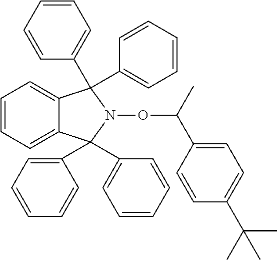 [SMILES: CC(ON1C(c2ccccc2)(c2ccccc2)c2ccccc2C1(c1ccccc1)c1ccccc1)c1ccc(C(C)(C)C)cc1]

A 100 ml flask was filled with argon and charged with dichloromethane (30 ml), 1,1,3,3-tetraphenylisoindoline-N-oxyl (4.39 g, 10 mmol, prepared as described in WO 2001/092228), 1-(1-bromoethyl)-4-tert-butylbenzene (2.89 g, 12 mmol, prepared as described by H. Kagechika, et al., Journal of Medicinal Chemistry, 32(5), 1098-108; 1989) and copper(I) bromide (2.87 g, 20 mmol). To the stirred suspension was added a solution of N,N,N′,N′,N″-pentamethyldiethylene-triamine (3.47 g, 20 mmol) in absolute ethanol (10 ml). The resulting green suspension was stirred 3 hours under argon and then additional (1-bromoethyl)-4-tert-butylbenzene (0.7 g, 2.9 mmol) was added. The green mixture was stirred at room temperature for 16 hours, then diluted with water (50 ml), and extracted with dichloromethane (3×30 ml). The combined extracts were washed with water (20 ml), 1M-HCl (2×20 ml), 1M-NH₃ (20 ml), and water (20 ml), and dried over MgSO₄. The residue was chromatographed on silica gel with heptane-ethyl acetate (50:1) and the pure fractions were crystallized from dichloromethane-methano to afford 5.6 g of the title compound as a white crystals. mp. 125-130° C. ¹H-NMR (400 MHz, CDCl₃. δ ppm): 7.6-6.7 (m, 28 ArH), 4.74-4.69 (q, J=4.8 Hz, —CHCH₃), 1.31 (s, C(CH₃)₃, 1.02-1.01 (d, J=4.8 Hz, —CHCH₃)

### Example 4

### Styrene Polymerization

1,3-Dihydro-1,1,3,3-tetraphenyl-2-(1-phenylethoxy)-1H-isoindol was mixed with styrene at ratios of 1:25, 1:50, 1:100, and 1:300 on a mol basis. Aliquots (0.2 ml) of each of the samples were then charged to low pressure/vacuum NMR (nuclear magnetic resonance) tubes. After charging, the NMR tubes were then sealed under nitrogen and heated to 70° C. to form a clear, soluble stock solution. The stock solutions were then refrigerated. Experiments were conducted by placing an NMR tube with the stock solution into an oil bath of desired temperature for a desired polymerization time. When the desired time was reached, the NMR tube was then rapidly cooled in an ice batch. NMR and GPC (gel permeation chromatograph) analyses were then conducted on the polymerization product of each tube.

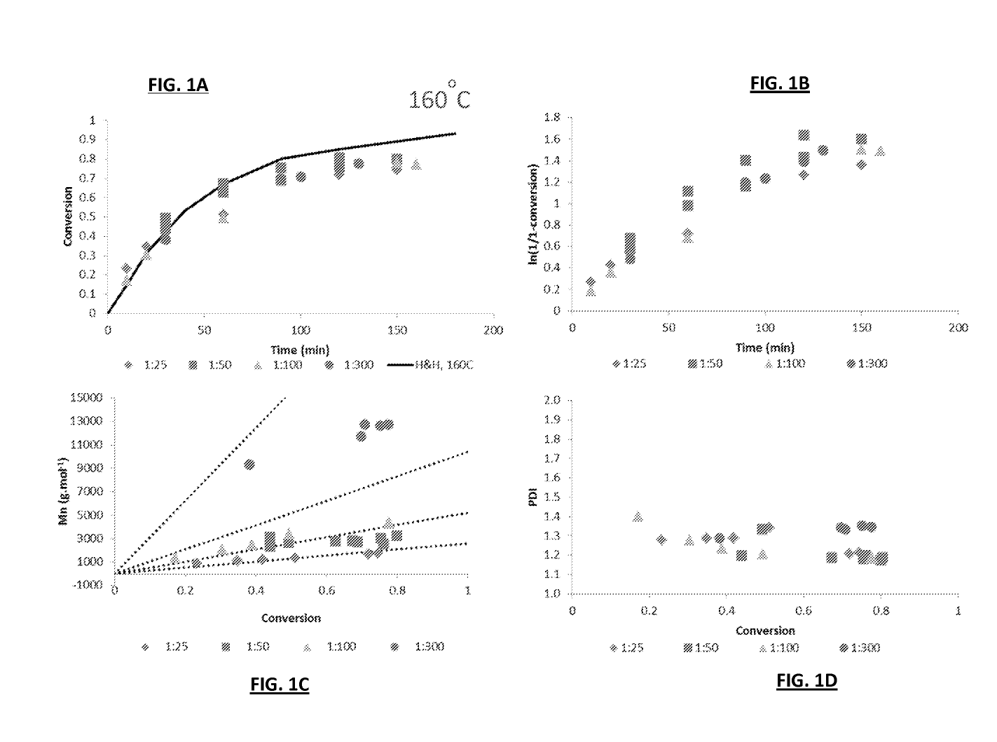

FIGS. 1A-D show that at 160° C., good control, low polydispersity and high reaction rate are obtained using 1,3-dihydro-1,1,3,3-tetraphenyl-2-(1-phenylethoxy)-1H-isoindol at a molar ratio from 1:25 to 1:300 based on the monomer. Experiments were repeated at higher temperatures. FIGS. 1A and 1B show high conversion rates across the various ratios of alkoxyamine:monomer. FIG. 1C is a graph showing the range of molecular weight distribution of the polystyrene formed in the reaction, reported as number average molecular weight (Mₙ; g/mol). FIG. 1D shows a relatively narrow polydispersity of the polystyrene from about 1.15 to about 1.4. This low polydispersity is substantially less than 1.5, which is the lowest possible value obtainable in non-controlled radical polymerizations (see e.g. Moad, G. et al. The Chemistry of Radical Polymerization, Elsevier 2006). Thus, the low polydispersity observed with the compounds of the present invention clearly indicates a controlled polymerization process. FIG. 1A shows that the reaction rate is comparable to that of bulk thermal polymerization of styrene, as reported by Hui et al. at 160° C. (Hui et al. J. App. Polym. Sci. 1972, 16, 749-769; indicated as H-H in the FIGs. for comparison). However, FIG. 1C demonstrates a linear change in molecular weight with conversion compared to bulk polymerization, which is indicative of controlled polymerization. FIG. 1D also confirms the low polydispersity with conversion, indicating good control of the polymerization process.

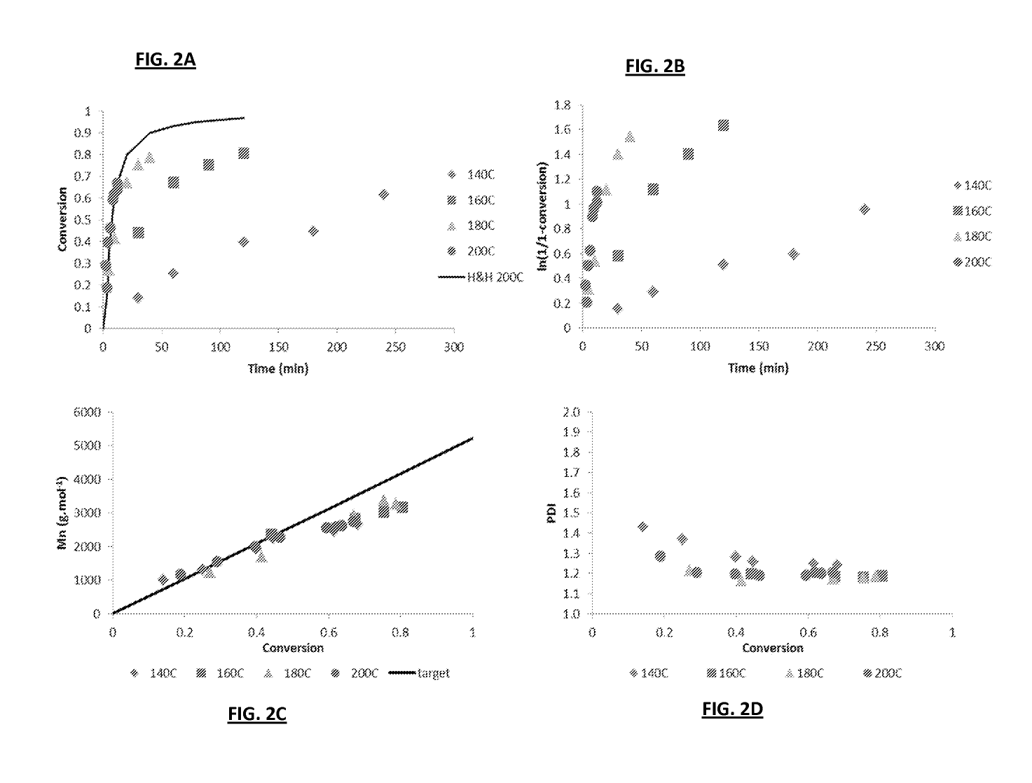

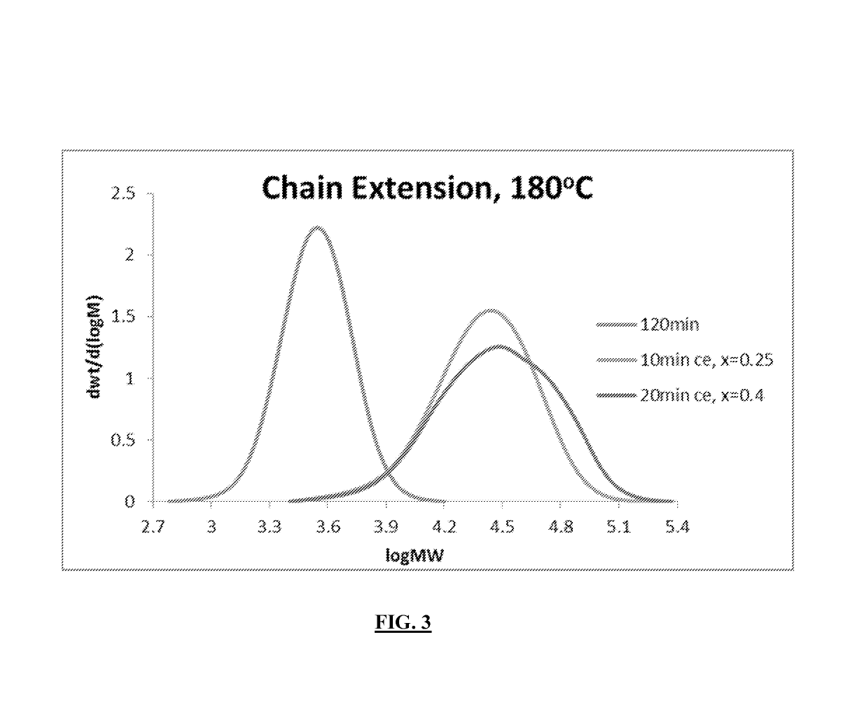

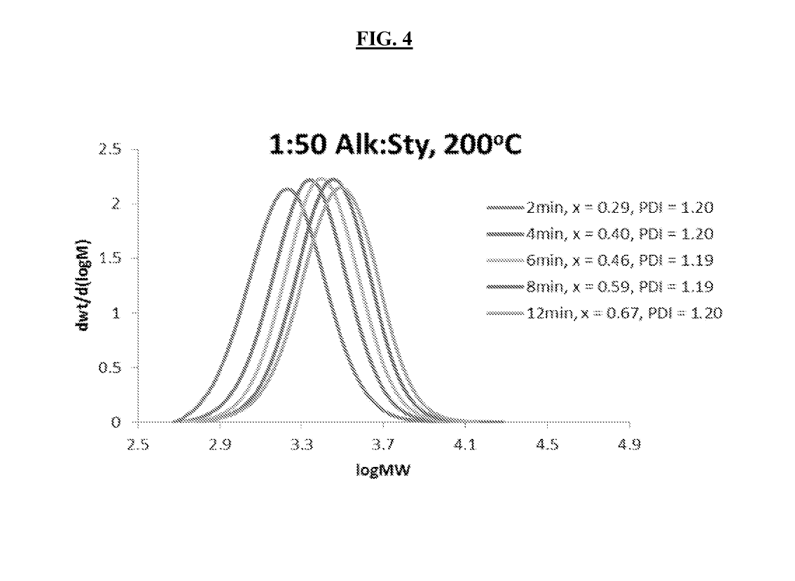

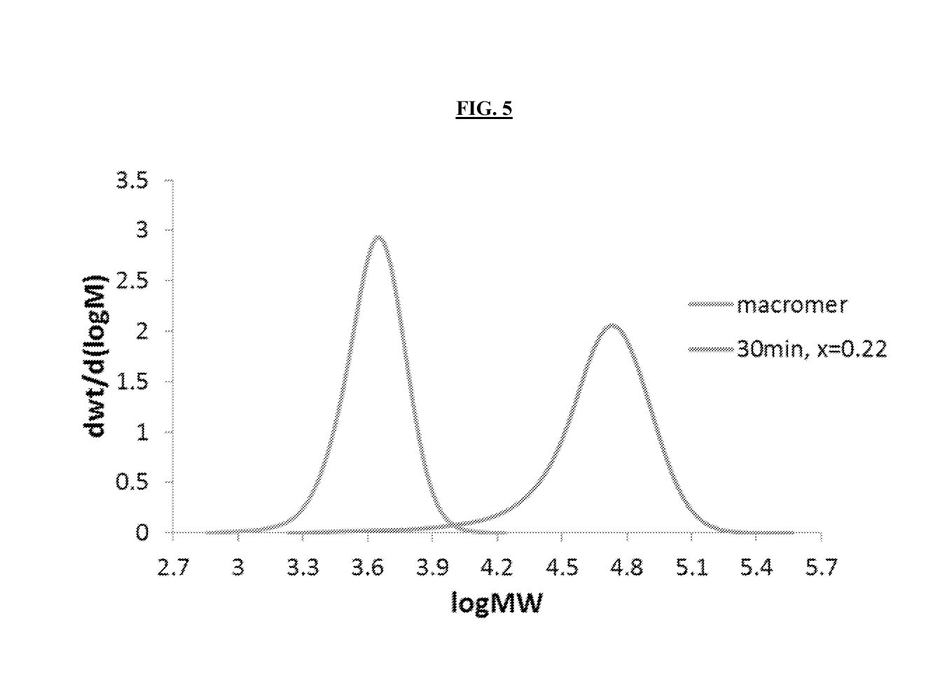

FIGS. 2A-D illustrate that even up to temperatures as high as 200° C., the 1,3-dihydro-1,1,3,3-tetraphenyl-2-(1-phenylethoxy)-1H-isoindol provides good control and low polydispersity indices. In each of FIGS. 2A-D, the ratio of 1,3-dihydro-1,1,3,3-tetraphenyl-2-(1-phenylethoxy)-1H-isoindol:monomer (styrene) was held constant at 1:50. As evidenced by the figures, the polydispersity index was as low as 1.15. The styrene polymerization rates are comparable to those obtained using conventional, bulk polymerizations. Good control of the polymerization for all experiments from 140° C. to 200° C. is evident by the linear change in number average molecular weight with conversion and the low polydispersity over the conversion range. At 200° C. high monomer conversions are reached in a few minutes with a linear increase in polymer chain-length with conversion, narrow molecular weight distribution and a final polydispersity index (PDI) of about 1.2 (FIG. 4). A sample from one of the experiments (160° C./1:50 Alk:Sty after 120 min) was charged with additional styrene and polymerized further. FIG. 3 shows that the number average molecular weight of the polymer molecular weight increased, indicating the polymer chains remained living after the first polymerization. This illustrates successful chain extension of the living polymer and that the methods may be used to prepare block polymers. A second chain extension experiment of polystyrene with a chain length 39 (produced at 160° C.; “macromer” in FIG. 5) was extended to 1226, as shown in FIG. 5. FIG. 5 illustrates that over an order-of-magnitude increase in number average molecular weight was achieved with no low number average molecular weight tail. It was determined the extended chain exhibited a polydispersity index ( [symbol] ) value of 1.4, thus demonstrating the high end-group functionality of the polystyrene macromer.

### Example 5

### Styrene/Alkoxyamines Block Co-Polymerization

Styrene and alkoxyamine at a molar ratio of 50:1 are to be fed to a first continuous stirred reactor (CSTR) at 200° C. with a 30 minute residence time. The reaction mixture is then to be continuously charged from the first CSTR to a second CSTR operating at the same conditions. After the second CSTR, the reaction product is to be mixed with butyl acrylate at a 1:2 molar ratio of butyl acrylate:styrene in a third CSTR to form a block styrene-butylacrylate co-polymer.

### Example 6

### Styrene/Alkoxyamine Block Co-Polymerization Tube Reactor

Styrene and alkoxyamine at a molar ratio of 50:1 are to be fed to a first tube reactor at 200° C. with a 30 minute residence time. The reaction mixture is then to be continuously charged from the first tube reactor to a second tube reactor where butyl acrylate is added to add a butylacrylate block to the styrene. After the second tube reactor, the reaction product is to be mixed with styrene in a third tube reactor to form a block styrene-butylacrylate-styrene co-polymer.

### Example 7

### Butyl acrylate polymerization with 1,3-dihydro-1,1,3,3-tetraphenyl-2-(1-phenylethoxy)-1H-isoindol at 160° C.

1,3-Dihydro-1,1,3,3-tetraphenyl-2-(1-phenylethoxy)-1H-isoindol was mixed with a 50/50 vol/vol mixture of butyl acrylate and dimethyl formamide solvent at a ratio of 1:50 on a mol basis between the isoindol and the butyl acrylate. Aliquots (0.2 ml) of each of the samples were then charged to low pressure/vacuum nuclear magnetic resonance (LPV NMR) tubes. After charging, the LPV NMR tubes were then sealed under nitrogen and heated to 70° C. to form a clear, soluble stock solution—no polymerization occurred at 70° C. as confirmed by ¹H NMR. The stock solutions were then refrigerated. Experiments were conducted by placing an LPV NMR tube with the stock solution into an oil bath at 160° C. After 1 hour the LPV NMR tube was then rapidly cooled in an ice batch. NMR and GPC (gel permeation chromatograph) analyses were then conducted on the polymerization product. After 1 hour, the butyl acrylate conversion was 65% and the polydispersity of the product was 1.53. The experiment was repeated by replacing 10 mol % of the butyl acrylate with styrene. After 1 hour, monomer conversion was 60% and the polydispersity of the product was 1.3. This example illustrates that the addition of a small amount of styrene to the acrylate polymerization can improve control of the polymerization.

### Example 8

### Butyl acrylate polymerization with 1,3-dihydro-1,1,3,3-tetraphenyl-2-(1-phenylethoxy)-1H-isoindol at varying temperatures

Another series of experiments using similar conditions as those in Example 7 were performed to evaluate the performance at different temperatures. 1,3-Dihydro-1,1,3,3-tetraphenyl-2-(1-phenylethoxy)-1H-isoindol was mixed with a 50/50 vol/vol mixture of butyl acrylate and dimethyl formamide solvent at a ratio of 1:50 on a mol basis between the isoindol and the butyl acrylate. Aliquots (0.2 ml) of each of the samples were then charged to LPV NMR tubes. The LPV NMR tubes were subjected to 4 freeze-pump-thaw cycles and sealed under nitrogen (<1 atm) using a Schlenk line and liquid nitrogen, to prevent monomer boiling during reaction at elevated temperature. The tubes were kept refrigerated until use and were suspended in a silicone oil bath to start the polymerization. The reaction was stopped at designated times by removing the tube and immersing in an ice bath for 30 seconds, with each tube used as an individual sample to reconstruct a complete polymerization profile.

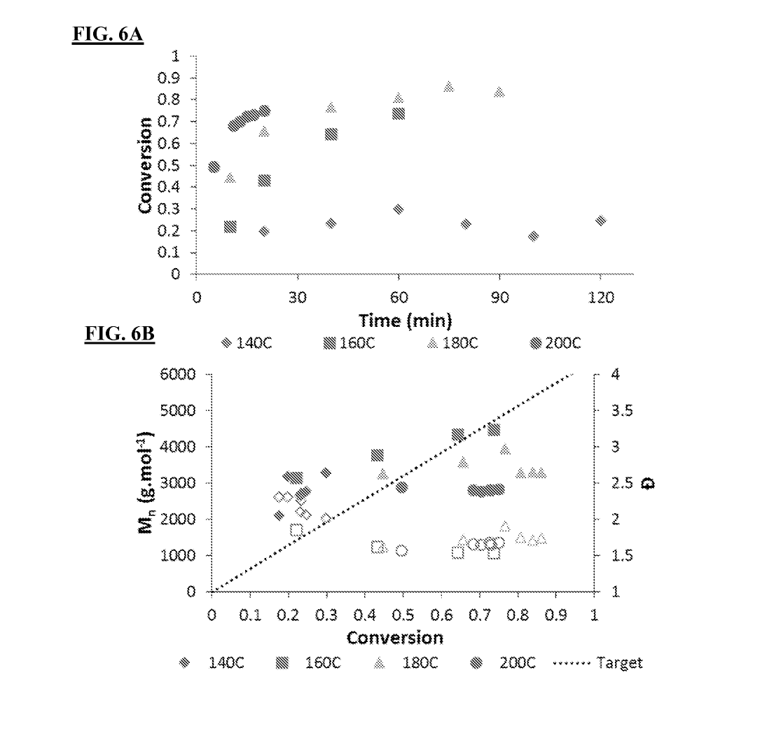

Conversion of the monomer over time at temperatures of 140° C., 160° C., 180° C., and 200° C. was evaluated (FIG. 6A). FIG. 6A illustrates that the polymerization rate increases with temperature up to the highest tested value of 200° C. With the decreased monomer content and the improved solubility due to inclusion of DMF, there is an improved dispersity at higher conversion with final values around 1.6 at 200° C. (FIG. 6B). In addition polymer Mₙ values decrease to below the target value, evidencing that thermal initiation of the monomer is a significant contribution to the total number of chains. ¹³C NMR of the products did not show any evidence of significant branching in bulk BA at 140° C. and 200° C.

### Example 9

### Styrene Polymerization with 2-[1-(4-tert-butylphenyl)ethoxy]-1,1,3,3-tetraphenyl-isoindoline at 160° C. with varying target chain lengths (TCL)

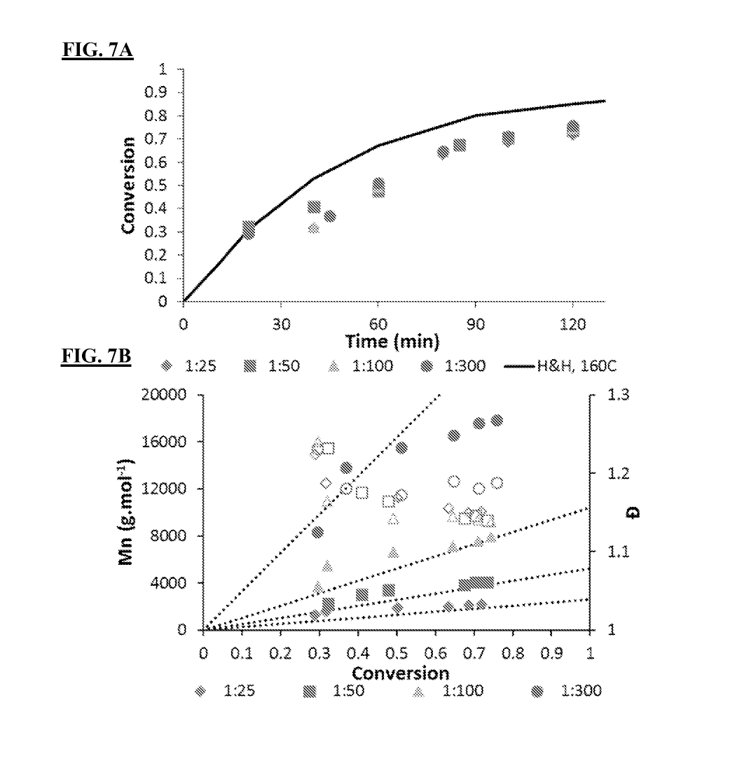

A range of alkoxyamine concentrations between 1:25 and 1:300 (on a mol basis between the alkoxyamine and the styrene) were used to generate polystyrene of varying target chain lengths (TCL) from the bulk monomer at 160° C. Note that longer target chain lengths are provided by lower concentrations of the alkoxyamine. Thus, the largest TCL corresponds to the lowest concentration of 2-[1-(4-tert-butylphenyl)ethoxy]-1,1,3,3-tetraphenyl-isoindoline tested (1:300), while the lowest TCL corresponds to the highest concentration tested. FIG. 7A is a plot of the monomer conversion profiles, and FIG. 7B illustrates the evolution of polymer chain length (as shown by the number-average molar masses (MO) and polydispersity ( [symbol] ) based on conversion, as compared to respective TCLs for each concentration, as illustrated by the dotted lines. The experimentally-measured polymer number-average molar masses are in excellent agreement with the target chain length across the range of alkoxyamine concentrations, with final polymer dispersities ( [symbol] ) of less than 1.2 (FIG. 7B).

### Example 10

### Styrene Polymerization with 2-[1-(4-tert-butylphenyl)ethoxy]-1,1,3,3-tetraphenyl-isoindoline at varying temperatures with a 1:50 mol ratio of alkoxyamine:styrene

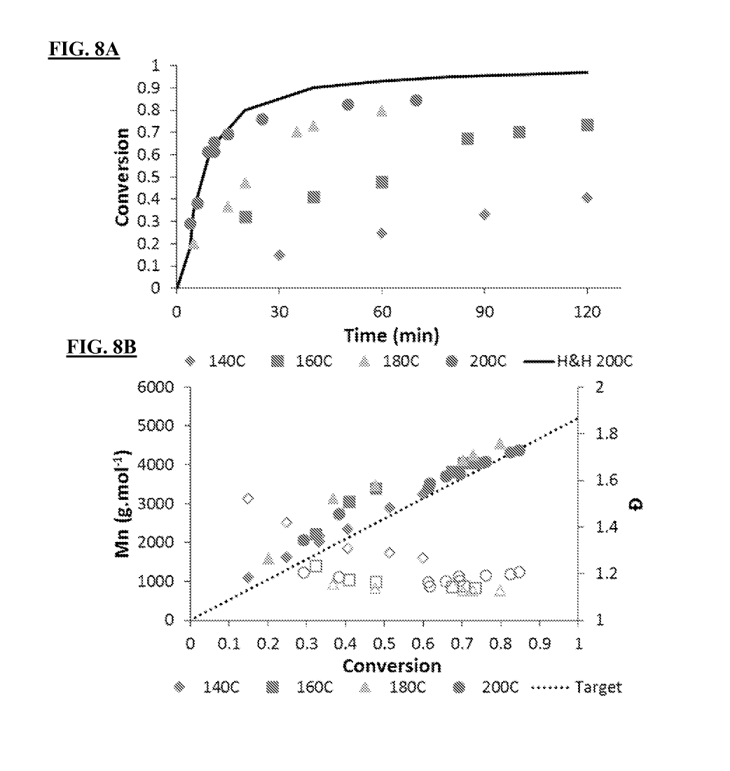

The stability of the nitroxide and efficacy of 2-[1-(4-tert-butylphenyl)ethoxy]-1,1,3,3-tetraphenyl-isoindoline as a mediating agent at elevated temperatures was further studied between 140° C. and 200° C. for a constant TCL. The polymerization rate accelerated with increasing temperature, with 70% conversion achieved in 15 minutes (FIG. 8A). The Mₙ profiles remain linear across the entire conversion range with [symbol] values at 1.15 for higher conversion (FIG. 8B), indicating that good control is maintained even at 200° C. Indeed, this unprecedented combination of fast reaction rate and excellent control indicates that the alkoxyamines of the presently claimed technology may be used at even higher temperatures.

### Example 11

### Butyl acrylate polymerization with 2-[1-(4-tert-butylphenyl)ethoxy]-1,1,3,3-tetraphenyl-isoindoline

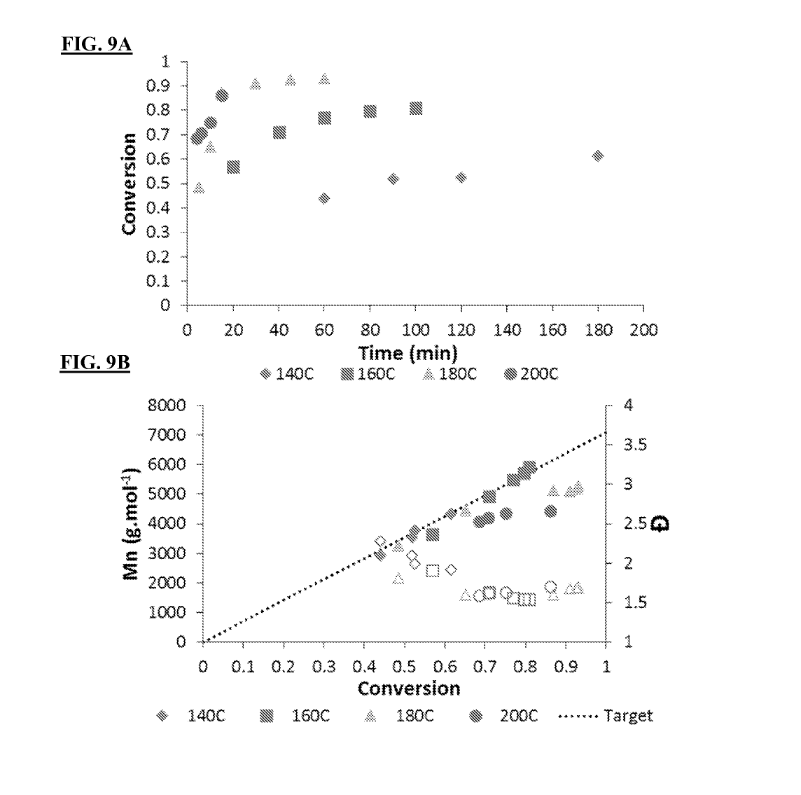

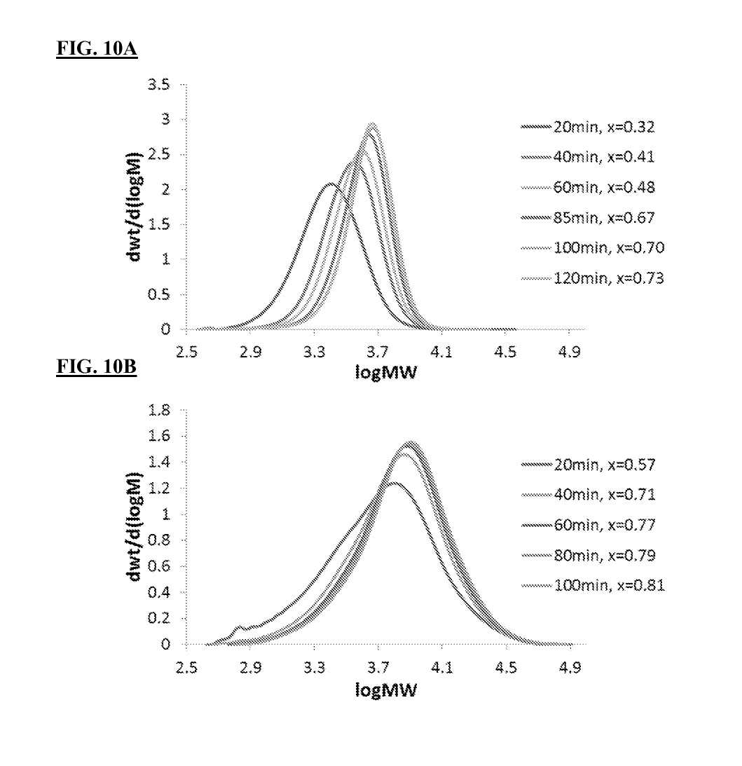

Unlike Examples 7 and 8, no solvent was utilized in the study of BA bulk homopolymerization by 2-[1-(4-tert-butylphenyl)ethoxy]-1,1,3,3-tetraphenyl-isoindoline over the same range of conditions examined for styrene. Results at varying temperatures for a constant TCL (1:55 alkoxyamine:butyl acrylate on a mol basis) are presented in FIG. 9. As shown in FIG. 9A, reaction rates in this system are even faster than those of styrene, with a monomer conversion of greater than 90% achieved in 15 minutes at 200° C. The number-average molecular weight (Mₙ) control remains good with the highest dispersity found at 140° C. (FIG. 9B). Without being bound by theory, this data suggests the alkoxyamine activation/deactivation kinetics are more favorable for control at higher temperatures, consistent with the data obtained for the polymerization of styrene by the same alkoxyamine. While final [symbol] values were 1.5-1.6, a result seen in the broader molar mass distributions of poly(butyl acrylate) (FIG. 10B) compared to polystyrene (FIG. 10A), this result is related to the significantly faster propagation kinetics of butyl acrylate compared to styrene. In addition, the molar mass distribution is broadened by the slower alkoxyamine initiation in the butyl acrylate system, as evidenced by the slowly disappearing peak at log(MW)=2.8 (FIG. 10B). Interestingly, no evidence of branching could be detected by ¹³C NMR at any of the temperatures, even for the poly(butyl acrylate) produced at 200° C. This result is consistent with other reversible deactivation radical polymerization processes. Without being bound by theory, it is hypothesized that fast deactivation suppresses the backbiting mechanism.

### Example 12

### Butyl acrylate polymerization with varying 2-[1-(4-tert-butylphenyl)ethoxy]-1,1,3,3-tetraphenyl-isoindoline concentrations at 160° C.

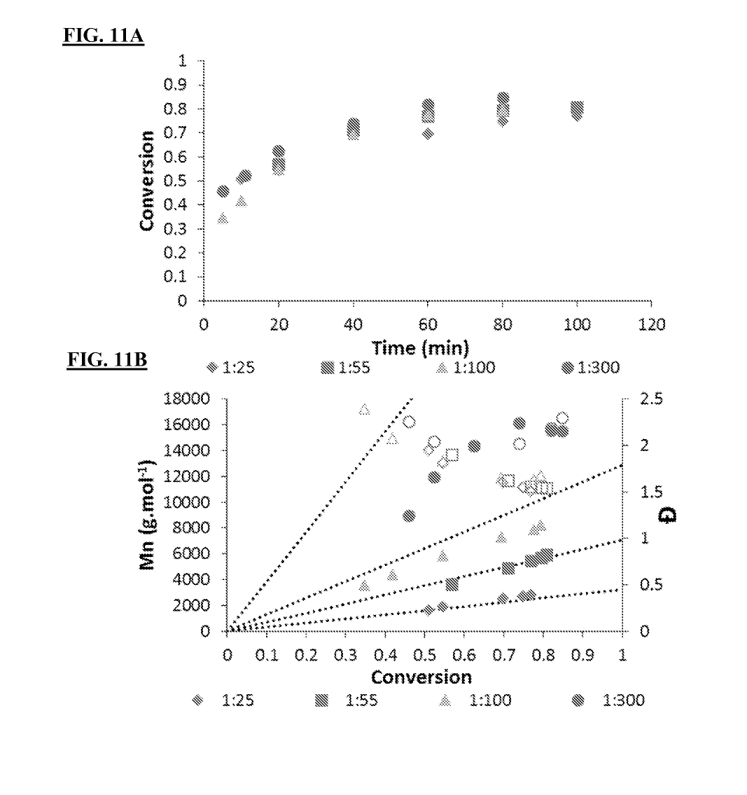

The TCL of butyl acrylate polymerizations in bulk were varied by varying the 2-[1-(4-tert-butylphenyl)ethoxy]-1,1,3,3-tetraphenyl-isoindoline concentration at 160° C. Results are provided in FIG. 11. Similar to the reaction with styrene, the change in concentration of 2-[1-(4-tert-butylphenyl)ethoxy]-1,1,3,3-tetraphenyl-isoindoline does not influence the rate of polymerization for the butyl acrylate system (FIG. 11A). Notably, there is adequate control of the polymerization with final [symbol] values around 1.5 (FIG. 11B), with the lowest concentration (1:300 alkoxyamine:butyl acrylate on a molar basis) being the only exception.

### Example 13

### Polymerization of additional monomers utilizing 2-[1-(4-tert-butylphenyl)ethoxy]-1,1,3,3-tetraphenyl-isoindoline

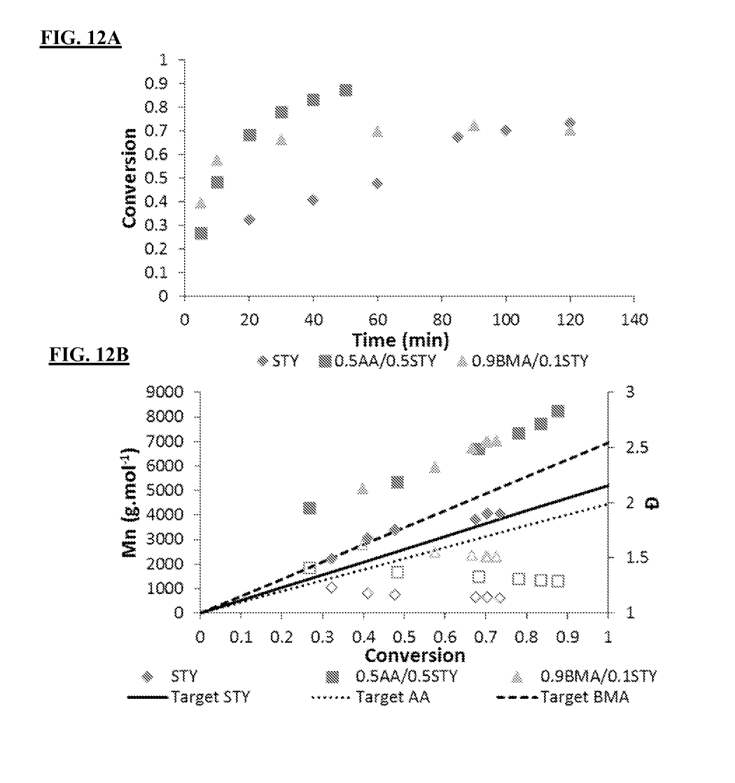

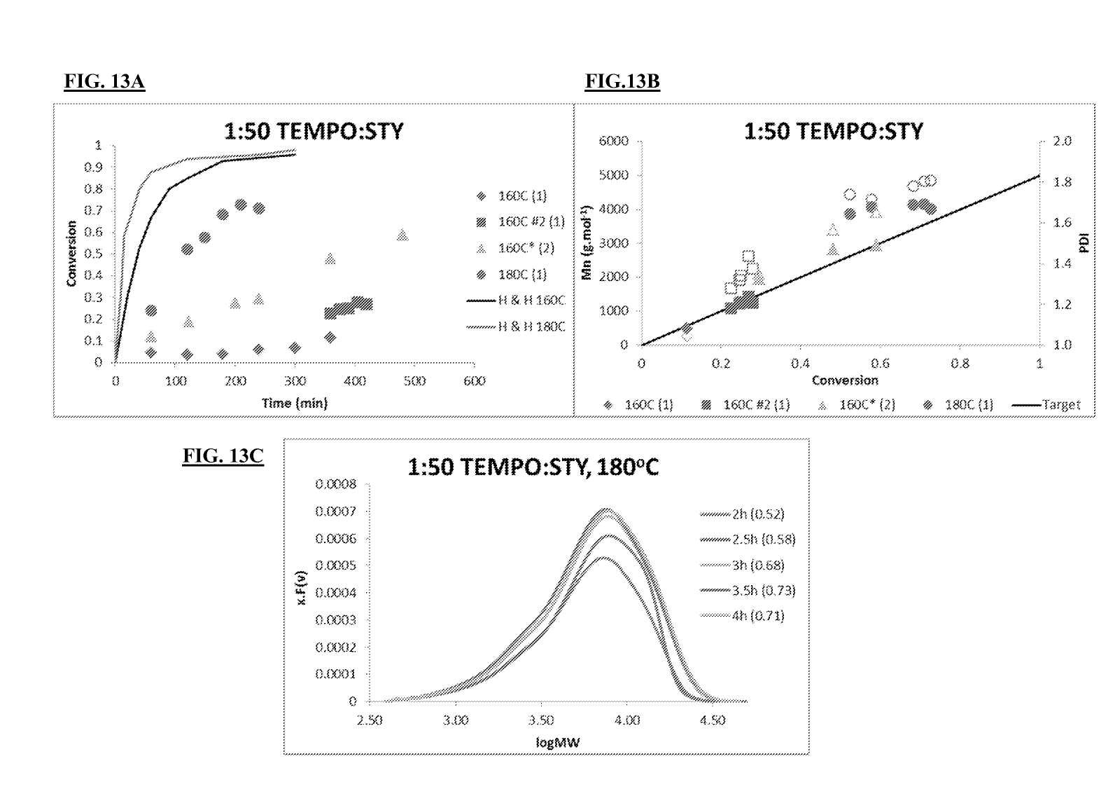

Other monomers were used to illustrate the range of monomer families that may be controlled with alkoxyamines of the present technology. FIG. 12 provides the results of using 2-[1-(4-tert-butylphenyl)ethoxy]-1,1,3,3-tetraphenyl-isoindoline with butyl methacrylate (BMA) and acrylic acid (AA) with 50 mol % styrene. The polymerization of butyl methacrylate with 10 mol % styrene depicts controlled polymerization, as evidenced by the linear increase in number average molecular weight and polydispersities around 1.5 (FIG. 13B). Surprisingly, the polymerization of acrylic acid with 50 mol % styrene exhibited an increased rate of polymerization (FIG. 13A) while concurrently maintaining good control of MW, with a final [symbol] of <1.3 (FIG. 13B). Thus, despite the high propagation rate coefficient of acrylic acid, excellent control is achieved for copolymerization of acrylic acid with styrene ( [symbol] =1.3 produced with 90% conversion in 60 min at 160° C.). FIG. 13B also evidences that control is achieved at 160° C. for bulk n-butyl methacrylate polymerized with 10 mol % STY.

### Comparative Example

Experiments were conducted with an alternative regulator (either TEMPO or 4-oxy-TEMPO; TEMPO is an abbreviation for 2,2,6,6-tetramethylpiperidin-1-yl)oxidanyl) in the same manner as above. FIGS. 13A-C shows that the rate of reaction at 160° C. and 180° C., are much lower than bulk polymerization. In addition, the number average molecular weight does not increase linearly with conversion above 160° C. FIG. 13C shows that a broad molecular weight distribution is found at elevated temperatures indicating lack of control. Without being bound by theory it is believed that in the bulk polymerization, the regulator (i.e. the TEMPO or the 4-oxy-TEMPO) is being decomposed.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A process of polymerizing a vinylic monomer, the process comprising:
combining a compound represented by Formula I with at least a first vinylic monomer to form a polymerization mixture; and
heating the polymerization mixture to a temperature that is about 130° C. or greater, and for a time sufficient to polymerize the vinylic monomer and form a first polymer;
wherein:
Formula I is: 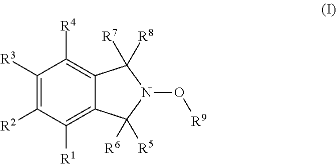
R¹, R², R³, and R⁴ are independently H, F, Cl, Br, I, CN, COOH, alkyl, cycloalkyl, alkoxy, alkylthio, C(O)O(alkyl), C(O)(alkyl), C(O)NH₂, C(O)NH(alkyl), C(O)N(alkyl)₂, or aryl, or R¹ and R², R² and R³, or R³ and R⁴ form together a 5- or 6-membered carbocyclic or heterocyclic ring;
R⁵, R⁶, R⁷, and R⁸ are independently aryl;
R⁹ is an unpaired electron, CR¹⁰R¹¹CN, CR¹⁰R¹¹(aryl), CR¹⁰R¹¹C(O)OH, CR¹⁰R¹¹C(O)O(alkyl), CR¹⁰R¹¹C(O)NH(alkyl), CR¹⁰R¹¹C(O)N(alkyl)₂, or CR¹⁰R¹¹C(O)(aryl); and
R¹⁰ and R¹¹ are independently H, alkyl, or together with the carbon to which they are attached they form a 5 or 6 membered carbocyclic ring.

2-3. (canceled)

4. The process of claim 1, wherein the temperature is about 160° C. to about 200° C.

5. (canceled)

6. The process of claim 1, wherein the first polymer has a polydispersity index from about 1.1 to about 1.6.

7-8. (canceled)

9. The process of claim 1, wherein the polymerization mixture further comprises a radical initiator that is a peroxide initiator or an azo-initiator.

10. (canceled)

11. The process of claim 1, wherein the first vinylic monomer comprises a styrenic monomer, an acrylate monomer, a methacrylate monomer, or a mixture of any two or more thereof.

12. (canceled)

13. The process of claim 1, wherein the first polymer is a first living polymer, the process further comprising adding at least a second vinylic monomer to the first living polymer, and heating to form a second living polymer.

14-17. (canceled)

18. The process of claim 13 further comprising adding at least a third vinylic monomer to the second living polymer, and heating to form a third living polymer.

19. (canceled)

20. The process of claim 1, wherein the first polymer has a number average molecular weight of from about 500 Daltons to about 100,000 Daltons.

21-23. (canceled)

24. The process of claim 1, wherein R¹, R², R³, and R⁴ are independently H, F, Cl, Br, I, CN, COOH, C₁-C₆ alkyl, C₅-C₆ cycloalkyl, C₁-C₆ alkoxy, C₁-C₆ alkylthio, C(O)O(C₁-C₆ alkyl), C(O)(C₁-C₆ alkyl), C(O)NH₂, C(O)NH(C₁-C₆ alkyl), C(O)N(C₁-C₆ alkyl)₂, or phenyl, or R¹ and R², R² and R³, or R³ and R⁴ form together a 5- or 6-membered carbocyclic or heterocyclic ring.

25. The process of claim 1, wherein R⁵, R⁶, R⁷, and R⁸ are independently phenyl, naphthyl, alkylphenyl, or alkylnaphthyl.

26. The process of claim 1, wherein R⁹ is an unpaired electron, CH₂Ph, CR¹⁰R¹¹CN, CH(CH₃)(aryl), C(CH₃)₂(aryl), CR¹⁰R¹¹C(O)OH, CR¹⁰R¹¹C(O)O(C₁-C₆ alkyl), CR¹⁰R¹¹C(O)(C₁-C₆ alkyl), CR¹⁰R¹¹C(O)NH(C₁-C₆ alkyl), CR¹⁰R¹¹C(O)N(C₁-C₆ alkyl)₂, CR¹⁰R¹¹CN, or CR¹⁰R¹¹C(O)Ph; R¹⁰ and R¹¹ are independently H, C₁-C₄ alkyl, or form together with the carbon to which they are attached a 5 or 6 membered carbocyclic ring; and aryl is phenyl or phenyl substituted with C₁-C₁₈ alkyl, O—C₁-C₁₈ alkyl, CN, —C(O)OH, —C(O)O(C₁-C₁₈ alkyl), F, Cl, Br, or I.

27-28. (canceled)

29. The process of claim 1, wherein R⁹ is

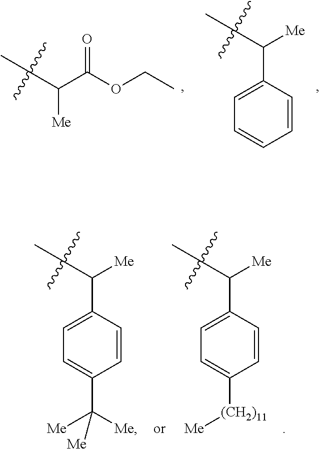

30. The process of claim 1, wherein the compound of Formula I is 1,3-dihydro-1,1,3,3-tetraphenyl-2-(1-phenylethoxy)-1H-isoindol, ethyl 2-((1,1,3,3-tetraphenylisoindolin-2-yl)oxy)propanoate, 2-[1-(4-dodecylphenyl)ethoxy]-1,1,3,3-tetraphenyl-isoindoline, or 2-[1-(4-tert-butylphenyl)ethoxy]-1,1,3,3-tetraphenyl-isoindoline.

31. The process of claim 1, wherein the compound of Formula I is subject to the provisos:
where if R¹, R², R³, and R⁴ are H and R⁹ is an unpaired electron or CHCH₃Ph, at least one of R⁵, R⁶, R⁷, and R⁸ is other than unsubstituted phenyl or where R⁹ is an unpaired electron or CHCH₃Ph, and R⁵, R⁶, R⁷, and R⁸ are unsubstituted phenyl, then at least one of R¹, R², R³, and R⁴ is other than H; and
where if R⁹ is an unpaired electron or CHCH₃Ph, and one of R⁵, R⁶, R⁷, and R⁸ is methyl and three of R⁵, R⁶, R⁷, and R⁸ are phenyl, then at least one of R¹, R², R³, and R⁴ is other than H.

32-38. (canceled)

39. A composition comprising the third living polymer formed by the process of claim 18.

40. The composition of claim 39 which is an adhesive, coating, plasticizer, pigment dispersant, compatibilizer, tackifier, surface primer, binder, or chain extender.

41-44. (canceled)

45. A compound represented by Formula I: 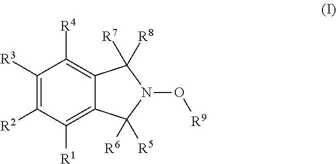
wherein:
R¹, R², R³, and R⁴ are independently H, F, Cl, Br, I, CN, C(O)OH, alkyl, cycloalkyl, alkoxy, alkylthio, C(O)O(alkyl), C(O)(alkyl), C(O)NH₂, C(O)NH(alkyl), C(O)N(alkyl)₂, or aryl, or R¹ and R², R² and R³, or R³ and R⁴ form together a 5- or 6-membered carbocyclic or heterocyclic ring;
R⁵, R⁶, R⁷, and R⁸ are aryl;
R⁹ is an unpaired electron, CR¹⁰R¹¹CN, CR¹⁰R¹¹(aryl), CR¹⁰R¹¹C(O)OH, CR¹⁰R¹¹C(O)O(alkyl), CR¹⁰R¹¹C(O)NH(alkyl), CR¹⁰R¹¹C(O)N(alkyl)₂, or CR¹⁰R¹¹C(O)(aryl); and
R¹⁰ and R¹¹ are independently H, alkyl, or together with the carbon to which they are attached they form a 5 or 6 membered carbocyclic ring;
with the proviso that where R¹, R², R³, and R⁴ are H and R⁹ is an unpaired electron or CHCH₃Ph, at least one of R⁵, R⁶, R⁷, and R⁸ is other than unsubstituted phenyl, or where R⁹ is an unpaired electron or CHCH₃Ph, and R⁵, R⁶, R⁷, and R⁸ are unsubstituted phenyl, at least one of R¹, R², R³, and R⁴ is other than H; and where R⁹ is an unpaired electron or CHCH₃Ph, and one of R⁵, R⁶, R⁷, and R⁸ is methyl and three of R⁵, R⁶, R⁷, and R⁸ are phenyl, at least one of R¹, R², R³, and R⁴ is other than H.

46-50. (canceled)

51. The compound of claim 45, wherein R⁹ is

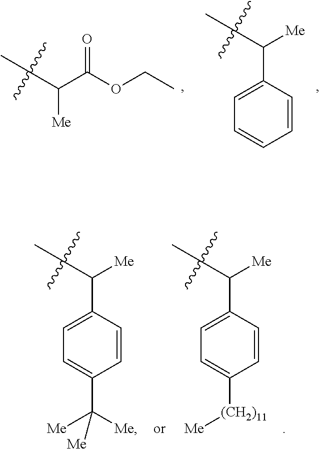

52. The compound of claim 45 which is 1,3-dihydro-1,1,3,3-tetraphenyl-2-(1-phenylethoxy)-1H-isoindol, ethyl 2-((1,1,3,3-tetraphenylisoindolin-2-yl)oxy)propanoate, 2-[1-(4-dodecylphenyl)ethoxy]-1,1,3,3-tetraphenyl-isoindoline, or 2-[1-(4-tert-butylphenyl)ethoxy]-1,1,3,3-tetraphenyl-isoindoline.

53. A process of polymerizing a vinylic monomer, the process comprising:
combining a compound of claim 45 with at least a first vinylic monomer to form a polymerization mixture; and
heating the polymerization mixture to a temperature that is about 130° C. or greater, and for a time sufficient to polymerize the vinylic monomer and form a first polymer.
